(19) United States
(12) Patent Application Publication
(10) Pub. No.: US 2021/0139834 A1
(43) Pub. Date: May 13, 2021

(54) METHODS AND COMPOSITIONS FOR FORMULATING AND DISPENSING PHARMACEUTICAL FORMULATIONS

(71) Applicant: PLURISTEM LTD., Haifa (IL)

(72) Inventors: Lior RAVIV, Kfar Monash (IL); Nadav ESHKOL, Haifa (IL); Dorina ROBERMAN, Nahariya (IL)

(73) Assignee: PLURISTEM LTD., Haifa (IL)

(21) Appl. No.: 17/047,411

(22) PCT Filed: Apr. 16, 2019
(86) PCT No.: PCT/IB2019/053115

§ 371 Date: Oct. 14, 2020

(30) Foreign Application Priority Data
Apr. 16, 2018 (IL) 258738

(51) Int. Cl. C12M 1/06; B01F 3/08; B01F 7/20; B01F 15/00; B01F 15/04; B01F 15/06; B65B 3/00; A61K 35/50; A61K 35/28; A61K 35/35; A61J 1/20; C12M 1/26; C12M 1/34; C12M 1/36

(52) U.S. Cl. CPC C12M 27/02; B01F 2215/0037; B01F 7/20; B01F 15/00175; B01F 15/0022; B01F 15/00285; B01F 15/0479; B01F 15/065; B65B 3/003; A61K 35/50; A61K 35/28; A61K 35/35; A61J 1/2003; C12M 33/00; C12M 41/36; C12M 41/46; C12M 41/26; C12M 41/32; C12M 41/48; B01F 2003/0896; B01F 2215/0032; B01F 3/0853

(57) ABSTRACT

The present disclosure relates to use of specialized vessels for formulating and dispensing pharmaceutical formulations. The vessels are optionally able to maintain homeostatic conditions and a homogeneous constitution of pharmaceutical suspensions, while simultaneously dispensing them into single-use containers.

### FIELD OF THE TECHNOLOGY

The present disclosure relates to use of specialized vessels for formulating and dispensing pharmaceutical formulations. The vessels are optionally able to maintain homeostatic conditions and a homogeneous constitution of pharmaceutical suspensions, while simultaneously dispensing them into aliquots (e.g. single-use containers).

### BACKGROUND

Final formulation of pharmaceutical suspensions is currently carried out in sealed containers. The resulting suspension may then be manually dispensed into aliquots (e.g. single-use containers), using a pump or the like. The inventors seek to add functionality and control to these processes, which is sorely needed in the art (Campbell et al., Nguyen et al., Pattasseril et al., Pigeau et al.).

### SUMMARY OF THE DISCLOSURE

Aspects of the disclosure relate to systems and methods that enable improved formulating and dispensing of pharmaceutical formulations.

Additional embodiments consistent with principles of the disclosure are set forth in the detailed description which follows or may be learned by practice of methods or use of systems or articles of manufacture disclosed herein. It is understood that both the foregoing general description and the following detailed description are exemplary and explanatory only, and are not restrictive of the disclosure as claimed. Additionally, it is to be understood that other embodiments may be utilized and that electrical, logical, and structural changes may be made without departing form the spirit and scope of the present disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure. In the drawings:

FIG. 1 is a perspective view of a system for growing and harvesting cells, according to an exemplary embodiment.

FIG. 2A depicts a schematic, cross-sectional view of a cylindrical, round-bottomed bioreactor 101 with agitation device (partially not depicted), where Dt indicates the inner tank diameter, Hi indicates the height of axial rod 102 of agitation device, and Di indicates the diameter of blades (not depicted) of agitation device. The distance between the Wb arrow-tips shows the width of baffles 103. B depicts a perspective view of bioreactor 47, showing additional features axial rod 41, baffles 42, blades 43, solution emptying tubing 44, tubing 45 for self-circulation of suspension, and exit tubing 46 for aliquot filling. C depicts a different side view of axial rod 41, baffles 42, and blades 43.

FIG. 3A is a perspective view of a carrier (or “3D body”), according to an exemplary embodiment. B is a perspective view of a carrier, according to another exemplary embodiment. C is a cross-sectional view of a carrier, according to an exemplary embodiment.

### DESCRIPTION OF THE EMBODIMENTS

Reference will now be made in detail to the present embodiments of the disclosure, examples of which are illustrated in the accompanying drawings.

In this application, the use of the singular includes the plural unless specifically stated otherwise. Also in this application, the use of “or” means “and/or” unless stated otherwise. Furthermore, the use of the term “including,” as well as other forms, such as “includes” and “included,” are not limiting. Any range described herein will be understood to include the endpoints and all values between the end points.

Provided herein, in certain embodiments, is a method of formulating a pharmaceutical product comprising an active agent, comprising: (a) introducing an initial liquid formulation, comprising the active agent, into a container; (b) agitating the initial liquid formulation; (c) determining the concentration of the active agent, enabling calculation of an amount of dilution solution necessary to achieve a target concentration of the active agent; and (d) adding the calculated amount of the dilution solution to the initial liquid formulation. Optionally, the thereby-diluted solution is further agitated. In certain embodiments, steps a-d are performed in the container; and the container is operably connected to (i) a portal(s) for aseptic transfer of a fluid material, e.g. a dilution solution, or final formulation, into and/or out of the container; and/or (ii) a means for controlling temperature inside the container. In other embodiments, the described container and portal are components of a closed system. Alternatively or in addition, the step of agitating the liquid (e.g. the initial liquid formulation, or in other embodiments the diluted formulation) serves to maintain homogeneity of the formulation. In other embodiments, the step of adding the dilution solution serves to generate a diluted liquid formulation, which is, in certain embodiments, an interim formulation, or is, in other embodiments, the final formulation.

Except where indicated otherwise, reference herein to a “final” formulation indicates a formulation that is suitable for administration to a subject.

Also provided herein is a method of formulating and dispensing a pharmaceutical product comprising an active agent, comprising: (a) introducing an initial liquid formulation, comprising the active agent, into a container; (b) agitating the initial liquid formulation; (c) determining a concentration of the active agent, enabling calculation of an amount of dilution solution necessary to achieve a target concentration of the active agent; (d) adding the amount of the dilution solution to the initial liquid formulation; and (e) repeatedly removing a predetermined volume of a fluid material from the container. In certain embodiments, the fluid material removed from the container is a volume of the final formulation. Optionally, the diluted solution is further agitated, prior to step (e). In certain embodiments, steps a-d are performed in a container that is operably connected to: (i) a portal(s) for aseptic transfer of a fluid material into and/or out of the container; and (ii) a means for controlling temperature inside the container. In other embodiments, the described container and portal are components of a closed system. Alternatively or in addition, the step of agitating the liquid (e.g. the initial liquid formulation, or in other embodiments the diluted formulation) serves to maintain homogeneity of the formulation. In other embodiments, the step of adding the dilution solution serves to generate a diluted liquid formulation, which is, in certain embodiments, an interim formulation, or is, in other embodiments, the final formulation.

Reference to a means for controlling temperature, except where indicated otherwise, refers to any means known in the art that is suitable for controlling the temperature of a receptable or liquid contents thereof, including, inter alia, (a) a thermometer and (b) a heater, cooler, or thermoelectric device for transferring heat to and/or from the receptacle or liquid. The terms “heater” and “cooler” are intended to be construed broadly to cover any device known in the art that is suitable for heating or cooling a liquid inside a closed container, preferably under sterile conditions. In certain embodiments, the liquid is kept under chilled conditions, e.g. 2-8° C., or, in other embodiments, 2-10° C., 2-7° C., 3-10° C., 3-8° C., or 3-6° C. In other embodiments, the liquid is kept under room-temperature conditions, e.g. 15-20° C., 15-22° C., 18-20° C., or 18-22° C. In still other embodiments, the liquid is kept under warmed conditions, e.g. 25-37° C., 25-35° C., 30-37° C., or 30-35° C. Each possibility represents a separate embodiment.

The term “pump” is intended to be construed broadly to cover any device known in the art that is capable of facilitating the flow of a fluid, or in other embodiments a gas, through a conduit.

The term “oxygen pump” is intended to be construed broadly to cover any device known in the art that is capable of facilitating the flow of oxygen through a conduit.

In some embodiments, the described interim formulation and/or final formulation comprise an excipient which is harmful or deleterious to cells subjected to an extended incubation in the presence of the excipient at non-chilled temperatures, for example an incubation of over 2 hours at temperatures above −20° C.; in in other embodiments an incubation of over 5, 4 or 3, hours, at temperatures over 4° C., 0° C., −10° C., or −30° C., where the incubation times and temperatures may be freely combined. Non-limiting examples of such excipients are DMSO (dimethyl sulfoxide, for example at concentrations of 3%-10%) and non-reducing disaccharides (e.g. trehalose and sucrose, for example at concentrations of 100 mM 1.5 M). Other, non-limiting examples of cryoprotectants are penetrating cryoprotectants such as glycerol and 1,2-propanediol, and non-penetrating cryoprotectants such as polyvinyl pyrrolidone, fructose, and glucose.

In certain embodiments, the systems described herein are closed systems. Alternatively or in addition, the described processes are automated processes. Those skilled in the art will appreciate in light of the present disclosure that closed systems are sealed from the outside environment, in a manner enabling maintenance of sterility. In further embodiments, closed systems are sealed in a manner preventing unintentional contamination by substances outside the system. In yet other embodiments, closed systems are sealed in an airtight manner. The skilled person will further appreciate that closed systems enable manipulation of the contents thereof without requiring the work to take place inside a sterile hood or other sterile environment.

In still other embodiments, the described methods and systems are fully sterilizable. Just methods and systems, in some embodiments, do not require a sterilization tank, a filter, or the like, when dispensing a formulation from the described container into aliquots.

In other embodiments, the described predetermined volume of a final formulation that may be repeatedly removed from the container is an aliquot volume. The term aliquot(s), except where indicated otherwise, refers to a formulation in a container for holding a pharmaceutical composition that is of a convenient size for administration to a subject. Non-limiting examples of such smaller containers are a vial, an ampule, a bag, or another vessel, e.g. a single-use vessel.

FIG. 1 is a perspective view of a system of the present disclosure, containing reservoirs 2-4 for active ingredient (2), liquid vehicle (3), and optional excipient(s) (4), which are optionally located on weight sensors (1). In certain embodiments, the excipient is a liquid excipient, a non-limiting example of which is DMSO. In other embodiments, the excipient is a stock solution of a dissolved or suspended excipient. In still other embodiments, e.g. in the case of cells as an active ingredient, the excipient may be a cryopreservant. The components of the initial formulation can be controllably moved through optional manifold valve 18, individually or in combination, through entry conduit 19 and into container/receptacle 20 via input port 28. In some embodiments, optional inbound pump 5 drives motion of the fluid through entry conduit 19. In other embodiments, the components of the initial formulation are premixed and added to a single reservoir (not depicted), which is moved by optional inbound pump 5 through entry conduit 19 into container/receptacle 20. Optional entry flow rate sensor 15 is configured to sense the flow rate into container/receptacle 20, for example by sensing the flow rate through entry conduit 19.

Once inside container/receptacle 20, initial formulation can be stirred with stirrer/agitation device 12, which may be operably connected with motor 11 via impeller shaft 25. The inner surface of walls 21 comprise one or, in other embodiments, multiple baffles 10, which jut inwards from the surface thereof and facilitate uniform mixing of initial formulation when stirrer/agitation device 12 is rotated. In more specific embodiments, 2-4 baffles are present, or in other embodiments, 1-2, 2-3, 1-3, 1-4, 2-5, 1-5, 2-5, or 3-5 baffles are present. Optionally, container/receptacle is surrounded, or in other embodiments partially surrounded, by insulation jacket 17.

When used for dispensing a pharmaceutical liquid, the system may utilize optional outbound pump 13 through exit port 29 and exit conduit 22 into the target container 14, which may be, in various embodiments, a vial, an ampule, a bag, or another vessel, e.g. a single-use vessel. Optional exit flow rate sensor 16 is configured to sense the flow rate out of container/receptacle 20, for example by sensing the flow rate through exit conduit 22.

The system further comprises temperature sensor 7. Additional, optional components include pH sensor 6, dO₂ sensor 8, and concentration sensor 9, which can be used to monitor conditions inside container/receptacle 20.

Container/receptacle 20 can include an upper cover or plate 23. In some embodiments, upper cover or plate 23 can be configured to seal container/receptacle 20. Upper cover or plate 23 can also include one or more additional ports 24. Container/receptacle 20 can also include other devices (not depicted) requiring access to container/receptacle 20.

In some embodiments, the described system is a converted bioreactor (not depicted). Those skilled in the art will appreciate that bioreactors comprise, in some embodiments, a container/receptacle 20 for containing medium; and control apparatus(es) 6-8, e.g. for sensing pH (6), temperature (7), and pO₂ (8) (e.g. of a solution or suspension disposed within the bioreactor), and optionally other parameters, e.g. the concentration(s) of glucose, lactate, lactate dehydrogenase, NH₃, and/or glutamate, each of which represents a separate embodiment. Alternatively or in addition, the bioreactor is configured to allow fluid exchange with external fluid reservoir(s) 2-4, or in other embodiments, spent medium container(s) (not depicted). For example, the bioreactor may contain port 28, for fluid inflow from external fluid reservoir(s) 2-4. In certain embodiments, the interior 27 of container/receptacle 20 does not contain any structures other than impeller shaft 25, stirrer/agitation device 12, and one or more baffles 10. In general, use of the term “bioreactor” herein to describe an apparatus is not necessarily intended to require that the apparatus was actually used for incubating cells under conditions compatible with cell expansion.

In yet other embodiments, any of the described methods further comprises facilitating uniform mixing of an initial formulation by rotation of a stirrer or agitation device. In still other embodiments, any of the described systems or apparatuses is configured to uniformly mix a final formulation when a stirrer/agitation device is rotated. In further embodiments, the container optionally further comprises 1 baffle; or in other embodiments 1-3 baffles; or, in other embodiments 2-3 baffles; or, in other embodiments 2-5 baffles; or in other embodiments more than 1 baffles, that jut(s) inward from an inward surface of the container.

Alternatively or in addition, the baffle width (the distance that the baffle juts into the receptacle) is between 0.03-0.2 of the container's widest diameter; or, in other embodiments, between 0.04-0.2, between 0.05-0.2, between 0.06-0.2, between 0.08-0.2, between 0.03-0.1, between 0.04-0.1, between 0.05-0.1, between 0.06-0.1, or between 0.08-0.1 of the container's widest diameter. As a non-limiting example, FIG. 2A depicts a cylindrical chamber with baffles whose width is 0.08 of the cylinder's diameter.

In certain embodiments, the described agitation device comprises a rotatable axial rod and blades connected to the bottom of the axial rod. In more specific embodiments, the blades have a diameter less than the container's widest diameter. In certain embodiments, for example in case of a medium receptacle with a concave bottom, use of blade with a smaller diameter facilitates mixing of the contents even when there is a small volume remaining. As a non-limiting example, FIG. 2A depicts a round-bottomed, cylindrical chamber illustrating this advantage, where the diameter of the blades is 0.6 of the inner tank diameter. In certain embodiments, the diameter of the blades is less than 80% of the container's diameter; or, in other embodiments, less than 70%, less than 60%, less than 50%, or less than 40% of the container's diameter; or, in other embodiments, between 50-80% of the container's diameter; or, in other embodiments, between 50-70%, 40-80%, 60-80%, 50-60%, 40-70%, or 40-60%. In certain embodiments, the aforementioned features are combined with baffles. As provided herein, such combinations provided significant advantages in formulating and aliquoting relatively large volumes (e.g. 1 liter or greater) of cell suspensions.

In certain embodiments, an aperture at the bottom of the described container or receptacle is utilized for removing a predetermined volume of the final formulation. A non-limiting example of these embodiments is depicted in FIG. 2B, which shows a perspective view of bioreactor 47, showing additional features axial rod 41, baffles 42, blades 43, solution emptying tubing 44, tubing 45 for self-circulation of suspension, and exit tubing 46 for aliquot filling. FIG. 2C depicts a different side view of axial rod 41, baffles 42, and blades 43 that have pear-shaped cross-section when viewed longitudinally, with a bulge in the lower half and tapering at the bottom.

The term initial liquid formulation, except where indicated otherwise, refers to a composition comprising a liquid carrier and an active pharmaceutical agent. Typically, the liquid carrier is an aqueous solution. In certain embodiments, the pharmaceutical agent is in suspension in the carrier. The term includes non-final formulations, for example formulations that require a dilution solution in order to reach the concentrations of the intended final formulation.

In other, optional embodiments, any of the described methods further comprises determining a concentration of the active agent in the suspension. Thus, the described container is optionally further operably connected to a sensor for determining a concentration of the active agent in the suspension. The active agent is, in some embodiments, in suspension. In other embodiments, the active ingredient is a particulate material. In more specific embodiments, the particulate material comprises living cells, or in other embodiments, inactivated cells. In other embodiments, the particulate material consists essentially of living cells, or in other embodiments, inactivated cells. In yet other embodiments, the particulate material consists of living cells, or in other embodiments, inactivated cells. In more specific embodiments, the cells may be adherent stromal cells. In yet more specific embodiments, the adherent stromal cells are placenta-derived. Alternatively, the adherent stromal cells are derived from adipose tissue, or in other embodiments, from bone marrow.

Alternatively or in addition, any of the described methods further comprises determining an average size of a particulate material in the suspension. Thus, the container is optionally further operably connected to a sensor for determining an average size of a particulate material in the suspension, which may be, in non-limiting embodiments, living cells, or in other embodiments, inactivated cells. In more specific embodiments, the cells may be adherent stromal cells. In yet more specific embodiments, the adherent stromal cells are placenta-derived. Alternatively, the adherent stromal cells are derived from adipose tissue, or in other embodiments, from bone marrow.

In still other embodiments, any of the described methods further comprises measuring the number of viable cells in a pharmaceutical suspension. In other embodiments, the described container is optionally further operably connected to a means of measuring the number of viable cells in a suspension inside the container. Viability of cells may be measured by a variety of methods known in the art, e.g. by removing a sample and counting the cells present therein, or by measuring the biomass using an electrode that measures capacitance (commercially available, for example Aber Instruments Biomass Monitor 230). Those skilled in the art will appreciate that the precise method of measuring the number of viable cells is not critical to carrying out the described methods.

In other embodiments, an electrode can be used to measure homogeneity of a suspension. For example, if repeated measurements are taken, the degree of oscillation in the capacitance, if any, reflects the degree of heterogeneity of the cell density in the suspension.

In yet other embodiments, any of the described methods further comprises monitoring and/or controlling pH of the initial liquid formulation. Thus, the container is optionally further operably connected to a means of monitoring and/or controlling pH of the initial liquid formulation. Those skilled in the art will appreciate, in light of the present disclosure, that the pH of a liquid formulation can be adjusted in a variety of ways known in the art, non-limiting examples of which are addition of carbon dioxide (CO₂), base solution, acid solution, and/or pH buffer to the formulation. Non-limiting examples of means for adjusting pH include pumps for addition of CO₂, base solution, acid solution, and/or pH buffer to the formulation. In certain embodiments, the described system comprises adjustable controls for the pH of the formulation.

In other embodiments, any of the described methods further comprises monitoring and/or controlling the dissolved oxygen concentration (pO₂) inside the container. Thus, the container is optionally further operably connected to a means of monitoring and/or controlling the pO₂ inside the container (e.g. of a solution or suspension disposed within the container). pO₂ can be adjusted (as a non-limiting example) by addition of 02 to a formulation, in some embodiments using a pump. In certain embodiments, the described system comprises adjustable controls for the pO₂ of the formulation; and/or other parameters, e.g. the concentration(s) of one or more of, in other embodiments 2 or more of, in other embodiments 3 or more of, in other embodiments 4 or more of, or in other embodiments all of glucose, lactate, lactate dehydrogenase, NH₃, and/or glutamate, each of which represents a separate embodiment. In still other embodiments, any of the aforementioned parameters, or any combination thereof, is kept constant during dispensing the described solution or suspension into external containers, e.g. despite volume changes in a solution or suspension inside the container. In certain embodiments, the parameter(s) are kept constant throughout a decrease in volume to 25% of the maximal volume of the container, or in other embodiments, 50%, 40%, 33%, 30%, 20%, 15%, 12%, 10%, 8% or 5% of the maximal volume of the container.

In yet other embodiments, the suspension (e.g. a cell suspension) is kept homogeneous during dispensing the described solution or suspension into external containers, e.g. despite volume changes in a solution or suspension inside the container. In certain embodiments, the suspension is kept homogeneous throughout a decrease in volume to 25% of the maximal volume of the container, or in other embodiments, 50%, 40%, 33%, 30%, 20%, 15%, 12%, 10%, 8% or 5% of the maximal volume of the container. “Homogenous”, except where indicated otherwise, indicates a Coefficient of Variance (C.V.) of less than 10% among aliquots filled by the described apparatus. In other embodiments, the C.V. is less than 15%, 12%, or 8%.

Except where indicated otherwise, the term constant refers to maintenance of the relevant concentration or parameter with sufficient invariance to satisfy the standard requirements of pharmaceutical regulations. In other embodiments, accepted levels of variance are within 30% of a target value; or in other embodiments within 50%, 40%, 25%, 20%, 15% or 10% of a target value.

In yet other embodiments, any of the described methods further comprises collecting and/or storing data on conditions inside the container. Thus, the container is optionally further operably connected to a means of collecting and/or storing data on conditions inside the container, which may comprise e.g. a sensor and/or an external device for recording data from said sensor. In certain embodiments, the data is used to generate a report. Such conditions can include e.g., the temperature of the container, or in other embodiments of a solution or suspension disposed therein; or the osmolarity of a solution or suspension disposed in the container; or the pO₂, e.g. of a solution or suspension disposed within the container); and/or other parameters, e.g. the concentration(s) of one or more of, in other embodiments 2 or more of, in other embodiments 3 or more of, in other embodiments 4 or more of, or in other embodiments all of glucose, lactate, lactate dehydrogenase, NH₃, and/or glutamate, each of which represents a separate embodiment.

In still other embodiments, any of the described methods further comprises collecting and/or storing data on transfer of fluid into and/or out of the container. Thus, the container is optionally further operably connected to a means of collecting and/or storing data on transfer of fluid into and/or out of the container. In certain embodiments, the data is used to generate a report.

In other embodiments, any of the described methods comprises adding a predetermined volume of a fluid material into the container. Thus, the container is optionally further operably connected to a means of adding a predetermined volume of a fluid material into the container.

In yet other embodiments, any of the described methods further comprises controlling the flow rate of fluid material transferred into the container. Thus, the container is optionally further operably connected to a means (e.g. a pump) for controlling a flow rate of fluid material transferred into the container.

In still other embodiments, the described container is, optionally, further operably connected to a means of calibrating the means for controlling temperature.

In other embodiments, the described container is, optionally, further operably connected to a means of calibrating an agitation device used to agitate a liquid formulation.

In still other embodiments, the described container is, optionally, further operably connected to a means of calibrating other components and/or sensors described herein and/or monitoring the failure of one, some, or all of these components, of which represents a separate embodiment.

In other embodiments, any of the described methods further comprises controlling a flow rate of a fluid material removed from the container.

In still other embodiments, any of the described methods further comprises repeating the step of determining a concentration of the active agent, after a portion of the fluid material—or in other embodiments, at least some of the fluid material—has been removed from the container or receptacle. For example, the concentration of cells in the remaining final formulation in the receptacle may be determined each 2-10 minutes during removal of the final formulation from the container.

Each of the described optional method steps and optional components represents a separate embodiment, and they may be freely combined, in various embodiments.

In certain embodiments, the described methods are aseptic methods.

Also provided herein is an enclosed system, comprising a receptacle, wherein: (a) the receptacle comprises an initial liquid formulation, the initial liquid formulation comprising a liquid vehicle and an active agent; (b) the receptacle further comprises a means of agitating the initial liquid formulation; (c) the receptacle is operably connected with a means of determining a concentration of the active agent; (d) the receptacle is further operably connected with a means (e.g. a conduit, optionally in combination with a pump) of aseptically adding a dilution solution to the receptacle; and (e) the receptacle is further operably connected with a means for controlling temperature inside the receptacle. Those skilled in the art will appreciate, in light of the present disclosure, that determining the concentration of the active agent enables, in some embodiments, calculation of an amount of dilution solution necessary to introduce, or add, to the initial formulation, to achieve a target concentration of the active agent. In certain embodiments, the enclosed system is temperature-controlled. In other embodiments, the enclosed system is configured such that fluid contained within its receptacle is temperature-controlled.

In certain embodiments, the step of agitating the liquid (e.g. the initial liquid formulation, or in other embodiments the diluted formulation) serves to maintain homogeneity of the formulation. In other embodiments, the step of adding the dilution solution serves to generate a diluted liquid formulation, which is, in certain embodiments, an interim formulation, or is, in other embodiments, the final formulation.

Except where indicated otherwise, the term enclosed system indicates that the internal space of the system is encased so as to be physically separated from outside contaminants. Those skilled in the art will appreciate in light of the present disclosure that enclosed systems may, in some embodiments, comprise a closed volume and/or be sealed from the outside environment, in a manner enabling maintenance of sterility. In further embodiments, enclosed systems are sealed in a manner preventing unintentional contamination by substances outside the system. In yet other embodiments, enclosed systems are sealed in an airtight manner. The skilled person will further appreciate that enclosed systems enable manipulation of the contents thereof without requiring the work to take place inside a sterile hood or other sterile environment.

In certain embodiments, any of the described systems is configured for, and/or is capable of, formulating and/or dispensing a pharmaceutical product. In other embodiments, the system is used for formulating and/or dispensing a pharmaceutical product. In still other embodiments, the system comprises a finally formulated pharmaceutical product. In certain embodiments, a final formulation is repeatedly removed from the receptacle, e.g. via a conduit, optionally in combination with a pump. Such removal may generate convenient aliquots.

In other embodiments, any of the described systems is, optionally, further operably connected to a sensor for determining a concentration of the active agent in the suspension. The active agent is, in some embodiments, in suspension. In other embodiments, the active ingredient is a particulate material. In more specific embodiments, the particulate material comprises living cells, or in other embodiments, inactivated cells. In other embodiments, the particulate material consists essentially of living cells, or in other embodiments, inactivated cells. In yet other embodiments, the particulate material consists of living cells, or in other embodiments, inactivated cells. In more specific embodiments, the cells may be adherent stromal cells. In yet more specific embodiments, the adherent stromal cells are placenta-derived. Alternatively, the adherent stromal cells are derived from adipose tissue, or in other embodiments, from bone marrow.

In still other embodiments, any of the described systems is, optionally, further operably connected to a sensor for determining an average size of a particulate material in the suspension, which may be, in non-limiting embodiments, living cells, or in other embodiments, inactivated cells. In more specific embodiments, the cells may be adherent stromal cells. In yet more specific embodiments, the adherent stromal cells are placenta-derived. Alternatively, the adherent stromal cells are derived from adipose tissue, or in other embodiments, from bone marrow.

In yet other embodiments, any of the described systems is, optionally, further operably connected to a means of measuring viability of living cells in a pharmaceutical suspension.

In other embodiments, any of the described systems is, optionally, further operably connected to a means of monitoring and/or controlling pH of the initial liquid formulation. Those skilled in the art will appreciate in light of the present disclosure that the pH of a liquid formulation can be adjusted in a variety of ways known in the art, non-limiting examples of which are addition of CO₂, base solution, acid solution, and/or pH buffer to the formulation. Non-limiting examples of means for adjusting pH include pumps for addition of CO₂, base solution, acid solution, and/or pH buffer to the formulation. In certain embodiments, the described system comprises adjustable controls for the pH of the formulation.

In yet other embodiments, any of the described systems is, optionally, further operably connected to a means of monitoring and/or controlling the dissolved oxygen concentration (pO₂) inside the container. pO₂ can be adjusted (as a non-limiting example) by addition of O₂ to a formulation, in some embodiments using a pump. In certain embodiments, the described system comprises adjustable controls for the pO₂ of the formulation.

In other embodiments, any of the described systems is, optionally, further operably connected to a means of collecting and/or storing data on conditions inside the container. In certain embodiments, the data is used to generate a report.

In other embodiments, any of the described systems is, optionally, further operably connected to a means of collecting and/or storing data on transfer of fluid into and/or out of the container. In certain embodiments, the data is used to generate a report.

In other embodiments, any of the described systems is, optionally, further operably connected to a means of adding a predetermined volume of a fluid material into the container.

In other embodiments, any of the described systems is, optionally, further operably connected to a means of controlling a flow rate of fluid material (e.g. a dilution solution) transferred into the container.

In other embodiments, any of the described systems is, optionally, further operably connected to a means of calibrating the means for controlling temperature.

In other embodiments, any of the described systems is, optionally, further operably connected to a means of calibrating an agitation device used to agitate the initial liquid formulation.

In other embodiments, any of the described systems is, optionally, further operably connected to a means of calibrating other components and sensors and/or monitoring failure of one, some, or all of these components, each of which represents a separate embodiment.

In other embodiments, any of the described systems is, optionally, further operably connected to a means of controlling a flow rate of a fluid material (e.g. a final formulation) removed from the container.

Each of the described embodiments of the features enabling maintaining pharmaceutical suspensions under homeostatic conditions, maintaining homogeneity of pharmaceutical suspensions, and/or dispensing pharmaceutical suspensions into aliquots may be freely combined with each other. Moreover, each of these embodiments may be freely combined with each of the basic bioreactor embodiments described herein.

In other embodiments, the container for containing medium is associated with a sensor (e.g. a scale; not depicted) capable of sensing the mass or volume contained therein, thus, providing a means to monitor the amount of fluid introduced thereto. In still other embodiments, an external fluid reservoir is associated with a sensor (e.g. a scale) capable of sensing the mass or volume contained therein, thus, providing a means to monitor the amount of fluid removed therefrom. Such monitoring is useful e.g. for purposes of calculating the extent to which the initial liquid formulation has been diluted.

In other embodiments, the container holds more than 1 liter; or, in other embodiments, more than 1.5, more than 2, more than 3, more than 4, more than 5, more than 6, more than 8, more than 10, more than 12, more than 15, or more than 20; or, in still other embodiments, between 1-50, 1-100, 1-200, 2-50, 2-100, 2-200, 3-50, 3-100, 3-200, 4-50, 4-100, 4-200, 5-50, 5-100, 5-200, 10-50, 10-100, or 10-200 liters. As provided herein, formulation and dispensing methods useful for relatively small volumes of pharmaceutical suspensions (e.g. cell suspensions) are not suitable for larger volumes.

In certain embodiments, the cells in the described pharmaceutical suspension were previously cultured on 3D carriers. The carriers may be, in more specific embodiments, selected from macrocarriers, microcarriers, or either. Non-limiting examples of microcarriers that are available commercially include alginate-based (GEM, Global Cell Solutions), dextran-based (Cytodex, GE Healthcare), collagen-based (Cultispher, Percell), and polystyrene-based (SoloHill Engineering) microcarriers. In certain embodiments, the microcarriers are packed inside the perfused bioreactor.

In some embodiments, the carriers in the perfused bioreactor are loosely packed, for example forming a loose packed bed, which is submerged in a nutrient medium. Alternatively or in addition, the carriers are fibrous carriers that comprise an adherent material. In other embodiments, the surface of the carriers comprises an adherent material, or the surface of the carriers is adherent. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. In more particular embodiments, the material may be selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen.

In some embodiments, with reference to FIGS. 3A-B, and as described in WO/2014/037862, published on Mar. 13, 2014, which is incorporated herein by reference in its entirety, the cells in the described pharmaceutical suspension were previously cultured on grooved carriers 230. In various embodiments, the carriers may be used following a 2D incubation (e.g. on culture plates or dishes), or without a prior 2D incubation. In other embodiments, incubation on the carriers may be followed by incubation on a 3D substrate in a bioreactor, which may be, for example, a packed-bed substrate or microcarriers; or incubation on the carriers may not be followed by incubation on a 3D substrate. Carriers 230 can include multiple two-dimensional (2D) surfaces 212 extending from an exterior of carrier 230 towards an interior of carrier 230. As shown, the surfaces are formed by a group of ribs 214 that are spaced apart to form openings 216, which may be sized to allow flow of cells and culture medium (not shown) during use. With reference to FIG. 3C, carrier 230 can also include multiple 2D surfaces 212 extending from a central carrier axis 18 of carrier 230 and extending generally perpendicular to ribs 214 that are spaced apart to form openings 216, creating multiple 2D surfaces 212. In some embodiments, carriers 230 are “3D bodies” as described in WO/2014/037862; the contents of which relating to 3D bodies are incorporated herein by reference.

In still other embodiments, the material forming the multiple 2D surfaces comprises at least one polymer. Suitable coatings may, in some embodiments, be selected to control cell attachment or parameters of cell biology.

In certain embodiments, further steps of purification or enrichment for ASC have been performed. Such methods include, but are not limited to, cell sorting using markers for ASC and/or, in various embodiments, mesenchymal stromal cells or mesenchymal-like ASC.

Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

In certain embodiments, the cells in the pharmaceutical suspension have been subjected to a harvesting process, following expansion, that comprises oscillation. In certain embodiments, the agitation is vibration, for example as described in PCT International Application Publ. No. WO 2012/140519, which is incorporated herein by reference. In certain embodiments, during harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator. Non-limiting examples of a protease plus a calcium chelator are trypsin, or another enzyme with similar activity, optionally in combination with another enzyme, non-limiting examples of which are Collagenase Types I, II, III, and IV, with EDTA. Enzymes with similar activity to trypsin are well known in the art; non-limiting examples are TrypLE™, a fungal trypsin-like protease, and Collagenase, Types I, II, III, and IV, which are available commercially from Life Technologies. Enzymes with similar activity to collagenase are well known in the art; non-limiting examples are Dispase I and Dispase II, which are available commercially from Sigma-Aldrich. In still other embodiments, the cells are harvested by a process comprising an optional wash step, followed by incubation with collagenase, followed by incubation with trypsin. In various embodiments, at least one, at least two, or all three of the aforementioned steps comprise agitation. Alternatively or in addition, the ASC are expanded using an adherent material in a container, which is in turn disposed within a bioreactor chamber; and an apparatus is used to impart a reciprocating motion to the container relative to the bioreactor chamber, wherein the apparatus is configured to move the container in a manner causing cells attached to the adherent material to detach from the adherent material. In more specific embodiments, the vibrator comprises one or more controls for adjusting amplitude and frequency of the reciprocating motion. Alternatively or in addition, the adherent material is a 3D substrate, which comprises, in some embodiments, carriers comprising a synthetic adherent material.

Those skilled in the art will appreciate that a variety of isotonic buffers and media may be used for formulation of pharmaceutical suspensions. Hank's Balanced Salt Solution (HBSS; Life Technologies) is only one of many buffers that may be used. Other, non-limiting examples of useful base media include Minimum Essential Medium Eagle, ADC-1, LPM (Bovine Serum Albumin-free), F10 (HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. In certain embodiments, DMEM is used. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be fetal bovine serum, or in other embodiments another animal serum. In still other embodiments, the medium is serum-free.

Alternatively or in addition, the medium may be supplemented by growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

It will also be appreciated that in certain embodiments, when the described ASC are intended for administration to a human subject, the cells and the culture medium (e.g., with the above-described medium additives) are substantially xeno-free, i.e., devoid of any animal contaminants e.g., mycoplasma. For example, the culture medium can be supplemented with a serum-replacement, human serum and/or synthetic or recombinantly produced factors.

Other embodiments of the disclosure will be apparent to those skilled in the art from consideration of the specification and practice of the present disclosure. It is intended that the specification and examples be considered as exemplary only, with a true scope and spirit of the disclosure being indicated by the following claims.

### REFERENCES

Campbell et al., Concise Review: Process Development Considerations for Cell Therapy. Stem Cells Translational Medicine 2015; 4:1155-1163.
Nguyen et al., Process automation in manufacturing of mesenchymal stromal cells. Transfusion 2016; 56; 26S-28S.
Pattasseril et al., Downstream Technology Landscape for Large-Scale Therapeutic Cell Processing. BioProcess International 11(3)s March 2013, pp. 38-46.
Pigeau et al., Commercial Scale Manufacturing of Allogeneic Cell Therapy. Frontiers in Medicine. August 2018; Volume 5; Article 233.

1. A method of formulating a pharmaceutical product comprising an active agent, said active agent comprising living cells, the method comprising:
a. introducing an initial liquid formulation, comprising said active agent, into a container;
b. agitating said initial liquid formulation;
c. determining a concentration of said living cells, enabling calculation of an amount of dilution solution necessary to achieve a target concentration of said living cells; and
d. adding said amount of said dilution solution to said initial liquid formulation, thereby generating a final formulation,
e. repeatedly removing a predetermined volume of said final formulation from said container,
wherein steps a-e are performed in said container; and said container is operably connected to:
(i) a portal for aseptic transfer of said dilution solution into said container;
(ii) a portal for aseptic transfer of said predetermined volume of said final formulation out of said container; and
(iii) an apparatus for controlling temperature inside said container.

2. The method of claim 1, wherein said living cells are in a suspension, and wherein said container is operably connected to a sensor for determining concentration of said living cells in said suspension.

3. (canceled)

4. The method of claim 1, wherein said container is operably connected to a sensor for measuring viability of said living cells.

5. The method of claim 1, wherein said container is operably connected to (a) sensor for monitoring pH of said initial liquid formulation and means of controlling said pH; and (b) a sensor for monitoring dissolved oxygen concentration inside said container and an oxygen pump for controlling said dissolved oxygen concentration.

6. (canceled)

7. The method of claim 1, wherein said container is operably connected to a first sensor for collecting data on conditions inside said container; and a second sensor for collecting data on transfer of fluid into and/or out of said container.

8. (canceled)

9. The method of claim 1, wherein said container is operably connected to a pump for controlling a flow rate of fluid material transferred into said container.

10. The method of claim 1, further comprising mixing said final formulation with an agitation device, wherein said agitation device comprises a rotatable axial rod and blades connected to its bottom, and wherein said blades have a diameter less than widest diameter of said container.

11. (canceled)

12. The method of claim 1, wherein said portal for aseptic transfer of said predetermined volume of said final formulation out of said container utilizes an aperture at a bottom of said container.

13. The method of claim 1, wherein said container further comprises a baffle that juts inward from an inward surface thereof.

14. The method of claim 1, further comprising a pump for controlling a flow rate of removal of said fluid material removed from said container.

15. The method of claim 1, wherein said method is aseptic.

16. The method of claim 1, further comprising repeating the step of determining a concentration of said living cells, after removal of some of said final formulation from said container.

17. An enclosed system, comprising a receptacle, wherein:
a. said receptacle comprises an initial liquid formulation, said initial liquid formulation comprising a liquid vehicle and an active agent, said active agent comprising living cells;
b. said system is configured for agitating said initial liquid formulation;
c. said system is configured for determining concentration of said living cells;
d. said receptacle is further operably connected with a conduit for aseptically introducing a dilution solution into said receptacle, thereby generating a final formulation;
e. said receptacle is temperature-controlled; and
f. said receptacle is further operably connected with a conduit for repeatedly removing a predetermined volume of said final formulation from said container.

18. The enclosed system of claim 17, wherein said living cells are in a suspension.

19. The enclosed system of claim 18, wherein said receptacle is further operably connected to a sensor for determining concentration of said living cells in said suspension.

20. The enclosed system of claim 17, wherein said receptacle is further operably connected to a sensor for measuring viability of said living cells.

21-25. (canceled)

26. The enclosed system of claim 17, wherein said system is configured for mixing said final formulation with an agitation device, wherein said agitation device comprises a rotatable axial rod and blades connected to its bottom, and wherein said blades have a diameter less than a widest diameter of said container.

27. (canceled)

28. The enclosed system of claim 17, wherein repeatedly removing a predetermined volume of said final formulation utilizes an aperture at a bottom of said container.

29. The enclosed system of claim 17, wherein said system is sterile.

30. The enclosed system of claim 17, wherein said container further comprises a baffle that juts inward from an inward surface thereof.
